(19) United States
(12) Patent Application Publication
(10) Pub. No.: US 2005/0209592 A1
(43) Pub. Date: Sep. 22, 2005

(54) Plug-type connection for releasably connecting two bodies

(76) Inventors: Fridolin Schlapfer, Glarus (CH); Martin Hess, Holstein (CH); Robert M. Rauker, Chester Springs, PA (US); Charles E. Geltz, Drexel Hill, PA (US)

Correspondence Address: JONES DAY, 222 EAST 41ST ST, NEW YORK, NY, 10017, US

(21) Appl. No.: 11/060,614

(22) Filed: Feb. 17, 2005

Related U.S. Application Data
(63) Continuation-in-part of application No. 10/291,386, filed on Nov. 12, 2002, now abandoned.
Application No. 10/291,386 is a continuation of application No. PCT/CH00/00263, filed on May 11, 2000.

(51) Int. Cl. A61B 17/58

(52) U.S. Cl. 606/60

(57) ABSTRACT

The present invention relates to a plug-type connection for releasably connecting two bodies wherein the first body includes a first connection zone having a plurality of coaxially aligned first connection segments and the second body includes a plurality of coaxially aligned second connection segments. The first connection segment includes at least one segment with a non-circular cross-section and at least one segment having an axially tapering cross-section. The second connection segment includes at least one segment with a non-circular cross section sized and configured for rotatively engaging the non-circular cross section of the first connection segment and at least one segment sized and configured to mate with the at least one tapering section, wherein the tapered section of the first connection segment and the mating section of the second connection segment form an axially non-positive engagement for guiding the first body into engagement with the second body. The axially tapering cross-section of the first connection segment is preferably made from a deformable material so that the axially tapering cross section deforms when it mates with the second connection segment.

### CROSS-REFERENCE TO RELATED APPLICATIONS

This application is a continuation-in-part of U.S. patent application Ser. No. 10/291,386 filed Nov. 12, 2002, which is a continuation of International Patent Application PCT/CH00/00263, filed May 11, 2000, the entire contents of which are expressly incorporated herein by reference thereto.

### FIELD OF THE INVENTION

The invention relates to a plug-type connection for releasably connecting two bodies and in particular to a detachable connection between a pedicle screw and a screw driver.

### BACKGROUND OF THE INVENTION

For a minimally invasive and endoscopic use of surgical instruments and implants it is extremely important to establish a firm connection between screw and screwdriver. With screws having large head dimensions firm connections are no problem.

Small-headed screws having hexagon sockets present problems due to the small dimensions. It is, however, possible to use a screwdriver with a conical hexagon insert bit. Another possibility consists in turning the hexagon insert bit of the screwdriver to such an extent that it is still possible, by using some degree of force, to insert the screwdriver into the hexagon socket. The main problems with these solutions consist in a reduced strength of the drive segment and in the risk that while being manipulated the screw may fall off the screwdriver and disappear in the depth of the wound.

The reduced driving force and the risk of loosing the screw in the wound may be avoided by means of an additional screw holding sleeve. This solution, however, may require the use of a second instrument, which makes the manipulation more difficult and causes additional expenditure for instruments. In addition, the screwdriver equipped with a screw holding sleeve requires more space, which entails a higher degree of soft-tissue damage.

The present invention relates to a connection between screw and screwdriver which is resistant to tensile strain, to impact, and the strength of which is not reduced as it is rotated. In an illustrative embodiment of the invention, the operating end of the screwdriver, which is inserted into the screw head, includes, in addition to the screwdriver's drive segment properly speaking, a segment that may be wedged within the screw head.

### SUMMARY OF THE INVENTION

The plug-type connection for releasably connecting two bodies according to the invention comprises on one of said bodies a first connection zone including a central axis and M connection segments extending coaxially to the central axis as well as a second connection zone, formed in the second body, including a central axis and N connection segments. The M or respectively N connection segments are coaxially arranged relative to their respective central axes and may be accomplished on any of the connection zones in the form of plugs and/or recesses, so that the first connection zone and the second connection zone form a plug-type connection extending coaxially to the central axis. In addition, the first connection zone comprises at least one connection segment having a non-circular cross-section and the second connection zone comprises at least one connection segment having a cross-section that is in positive engagement with said non-circular cross-section. Thus it is possible to achieve a rotationally stabilized, positive engagement relative to the central axis. In addition, the plug-type connection provides a non-positive engagement which is effective coaxially to the central axis. Furthermore, concerning the number of connection segments, the following conditions are applicable:
M≧1
N≧1
M−N≧2

The first connection segments may be arranged coaxially and differ from each other in shape in the different embodiments of the device according to the invention. These differences in shape may derive from different cross-sections, such as hexagon, star-shaped form with six branches, or area of a circle, or else from different diameters with identical cross-sectional shape. The same is true of the second connection segments of the second connection zone formed in the second body.

In addition to the connection segments permitting a rotationally positive engagement of the two bodies, the plug-type connection according to the invention as realized in further embodiments comprises connection segments which cooperate in such a way as to cause an axially non-positive engagement, and, in yet another embodiment, also permit a stabilization of the central axis of the plug-type connection, which is of particular interest in the case of a non-positive engagement that is exposed to force. As to the arrangement of the connection segments, Tables 1-6 show non-limiting and illustrative variants of the invention.

TABLE 1

| | Connection zone 1 | | | Connection zone 2 | | |
|---|---|---|---|---|---|---|
| | Connection segments: M = 1 | | | Connection segments: N = 2 | | |
| | Segment with | Segment with | | Segment with | Segment with | |
| | positive | non-positive | Stabilization | positive | non-positive | Stabilization |
| M × N = 2 | engagement | engagement | segment | engagement | engagement | segment |
| Function: | | | | | | |
| Positive engagement | X | | | X | | |
| Non-positive engagement | X | | | (X) | X | |
| Stabilization | X | | | X | | |

TABLE 2

| | Connection zone 1 | | | Connection zone 2 | | |
|---|---|---|---|---|---|---|
| | Connection segments: M = 2 | | | Connection segments: N = 1 | | |
| | Segment with | Segment with | | Segment with | Segment with | |
| | positive | non-positive | Stabilization | positive | non-positive | Stabilization |
| M × N = 2 | engagement | engagement | segment | engagement | engagement | segment |
| Function: | | | | | | |
| Positive engagement | X | | | X | | |
| Non-positive engagement | (X) | X | | X | | |
| Stabilization | X | | | X | | |

TABLE 3

| | Connection zone 1 | | | Connection zone 2 | | |
|---|---|---|---|---|---|---|
| | Connection segments: M = 2 | | | Connection segments: N = 2 | | |
| | Segment with | Segment with | | Segment with | Segment with | |
| | positive | non-positive | Stabilization | positive | non-positive | Stabilization |
| M × N = 4 | engagement | engagement | segment | engagement | engagement | segment |
| Function: | | | | | | |
| Positive engagement | X | | | X | | |
| Non-positive engagement | (X) | X | | (X) | X | |
| Stabilization | X | | | X | | |

TABLE 4

| | Connection zone 1 | | | Connection zone 2 | | |
|---|---|---|---|---|---|---|
| | Connection segments: M = 2 | | | Connection segments: N = 3 | | |
| | Segment with | Segment with | | Segment with | Segment with | |
| | positive | non-positive | Stabilization | positive | non-positive | Stabilization |
| M × N = 6 | engagement | engagement | segment | engagement | engagement | segment |
| Function: | | | | | | |
| Positive engagement | X | | | X | | |
| Non-positive engagement | (X) | X | | (X) | X | (X) |
| Stabilization | X | | | X | | X |

TABLE 5

| | Connection zone 1 | | | Connection zone 2 | | |
|---|---|---|---|---|---|---|
| | Connection segments: M = 3 | | | Connection segments: N = 2 | | |
| | Segment with | Segment with | | Segment with | Segment with | |
| | positive | non-positive | Stabilization | positive | non-positive | Stabilization |
| M × N = 6 | engagement | engagement | segment | engagement | engagement | segment |
| Function: | | | | | | |
| Positive engagement | X | | | X | | |
| Non-positive engagement | (X) | X | (X) | (X) | X | |
| Stabilization | X | | X | X | | |

TABLE 6

| | Connection zone 1 | | | Connection zone 2 | | |
|---|---|---|---|---|---|---|
| | Connection segments: M = 3 | | | Connection segments: N = 3 | | |
| | Segment with | Segment with | | Segment with | Segment with | |
| | positive | non-positive | Stabilization | positive | non-positive | Stabilization |
| M × N = 9 | engagement | engagement | segment | engagement | engagement | segment |
| Function: | | | | | | |
| Positive engagement | X | | | X | | |
| Non-positive engagement | (X) | X | (X) | (X) | X | (X) |
| Stabilization | X | | X | X | | X |

Variants with M × N = 3 are equally possible.

The inventive device presenting one of the plug-type connections comprises as a first body a manipulation device, particularly a screwdriver, including the first connection zone, and as a second body a screw or an osteosynthetic implant, particularly a pedicle screw, including the second connection zone so that, in use, for example, the present invention may be used in an orthopedic operation to secure a patient's spine. That is, preferably the first body is a screwdriver and the second body is a pedicle screw so that in use, the screwdriver may be used to attach one or more pedicle screws to a patient's vertebrae so that a longitudinal spinal rod may be connected thereto to align the vertebrae. The present invention ensures that the pedicle screw will not separate from the screwdriver during installation.

In another embodiment of the device according to the invention the first and the second connection zones are interchanged, so that the manipulation device, in particular the screwdriver, comprises the second connection zone and, vice versa, the screw comprises the first connection zone.

The screwdriver is provided, beside the screwdriver drive segment (e.g. hexagon insert bit) representing a first connection segment, with a frusto-conical segment (screwdriver clamping segment) representing a second connection segment. Complementarily, the drive segment of the screw (e.g. hexagon socket) representing the first connection segment, is additionally provided with a screw clamping segment representing the second connection segment. For reasons of production technology, this screw clamping segment is preferably shaped in the form of a cylindrical bore having a diameter smaller than the maximum diameter of the frusto-conical screwdriver clamping segment and larger than the respective minimum diameter. The angle of the truncated cone is selected sufficiently small (2 to 10 degrees) so that on pressing the frusto-conical clamping segment of the screwdriver into the cylindrical clamping segment of the screw it becomes wedged therein and is retained by friction. This process of becoming wedged has created a connection between screwdriver and screw which is characterized by a good tensile strength without impairing the force of the rotative drive segment. Moreover, preferably, the frusto-conical segment may be made from a deformable material, such as, for example, an elastomeric or polymeric material such as silicone rubber, polyurethane, etc. so that, in use, as the frusto-conical segment of the screw driver is inserted into the screw clamping segment of the screw, the frusto-conical segment can deform thereby increasing the overall friction of the wedge-type connection. The remaining connection segments formed on the screw driver and screw may be made from any bio-compatible material known in the art including, but not limited to, stainless steel, titanium, titanium alloy, etc.

Wedge-shaped connections suffer from the disadvantage that the connection may abruptly come loose when exposed to bending stress. In the present case, however, the risk of the wedge-type connection coming loose under bending stress is reduced by the action of the rotative drive segment: the greater the length of the drive segment is, the greater will be the stabilizing effect.

For reasons of production technology, the drive segment of the screwdriver will preferably fit in the drive segment of the screw with a certain play and the stabilizing effect on the wedge-type connection becomes smaller as the play becomes greater. The negative influence of the play may be eliminated by completing the drive segment of the screwdriver with a cylindrical stabilization segment representing a third connection zone and by modifying the screw head correspondingly. From the point of view of production technology, the two cylindrical, mating stabilization segments may readily be provided with a tight fit.

Another possibility of reducing the negative influence of the play in the area of the drive segment consists in adding to the frusto-conical screwdriver clamping segment a cylindrical stabilization segment having a diameter smaller than or equal to the minimum diameter of said frusto-conical screwdriver clamping segment. In a complementary way, the cylindrical screw clamping segment must accordingly be lengthened by adding a cylindrical screw stabilization segment. From the point of view of production technology, the two cylindrical, mating stabilization segments may present a tight fit.

In a further embodiment, the screwdriver comprises an axially resilient pressure pin which extends coaxially to its longitudinal axis. After the screw has been inserted, the pressure pin is pushed forward relative to the screwdriver and pressed against the screw. Thus, the screwdriver may be entirely pressed out of the screw head. It is possible, by means of the pressure pin, to overcome even a strong compressive force generated by the automatic locking of the wedge-type connection and thus to release the connection between screwdriver and screw.

In cases in which the posterior column is operated on using an anterior approach, there may be a need for very long screwdrivers. Due to the great length of the screwdriver, even a slight pressure exerted by the surrounding soft tissues may cause problems during the removal of the screwdriver, arising from the screwdriver getting wedged in the drive segment of the screw. Using the ejection mechanism, the screwdriver may be removed even when exposed to lateral pressure exerted by the surrounding soft tissues.

Preferably, the invention makes it possible:

to establish a shock-resistant connection between screwdriver and screw;

to avoid an impairment of the driving force;

to require only a minimum space (no screw holding sleeve necessary); and

to resolve the problem, due to the ejection mechanism, of the screwdriver getting wedged in the drive segment of the screw.

### BRIEF DESCRIPTION OF THE DRAWINGS

Preferred features of the present invention are disclosed in the accompanying drawings, wherein similar reference characters denote similar elements throughout the several views, and wherein:

FIG. 1 shows a perspective view of a tenon-like connection zone according to an illustrative embodiment of the invention;

FIG. 2 shows a perspective view of a connection zone shaped in the form of a recess according to one embodiment of the inventive, plug-type connection;

FIGS. 3a) to d) are views showing local sections of different embodiments of the plug-type connection according to the invention;

FIGS. 4a) and b) are sectional views of connection zones which contain only recesses as connection segments, according to different embodiments of the invention;

FIGS. 5a) and b) are sectional views of connection zones which contain only tenon-like segments as connection segments, according to different embodiments of the invention;

FIG. 6 shows a table representing the possibilities of combining the connection zones according to FIGS. 4a) and b) with the connection zones according to FIGS. 5a) and b);

FIG. 7 shows perspective views of other connection zones according to different embodiments of the invention in which connection segments containing recesses are combined with connection segments containing tenon-like segments;

FIG. 8 is a perspective view of one embodiment of the device according to the invention; and

FIG. 9 is a perspective view of the pin serving for pushing away the screw according to the embodiment of the invention of FIG. 8.

### DETAILED DESCRIPTION OF THE PREFERRED EMBODIMENTS

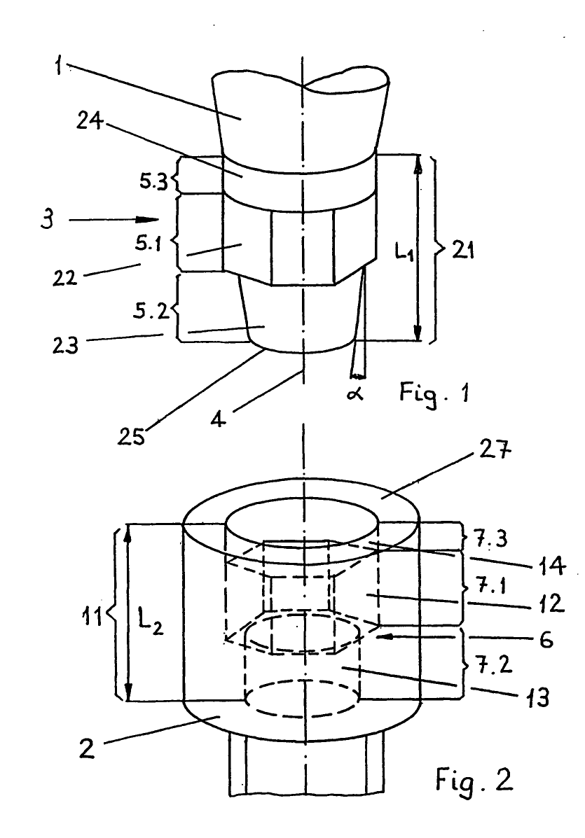

FIGS. 1 and 2 represent an embodiment of the plug-type connection according to the invention which comprises a first body 1 having a first connection zone 3 extending concentrically to a central axis 4 and a second body 2 having a connection zone 6 which equally extends concentrically to said central axis 4. Adjoining its front end 25, the body 1 comprises the first connection zone 3 including three (M=3) connection segments 5.1; 5.2; 5.3, which axially adjoin one another, beginning from the front end 25, and extend over a length L₁. The connection zone 3 is shaped in the form of a shaft segment 21 extending concentrically to the central axis 4. The first connection segment 5.1 with a non-circular cross section Q_{5.1} is shaped in the form of a hexagonal segment 22 and has a width across corners E and a width across flats SW, whereas the first connection segment 5.2 designed for establishing an axially non-positive connection is shaped in the form of a frusto-conical shaft segment 23 having a cone angle of between 2 and 10 degrees, preferably between 3 and 5 degrees. The frusto-conical shaft segment 23 adjoins the front end 25 and, in this portion, has a diameter dₘᵢₙ while in the portion adjoining the hexagonal segment 22 it has a diameter dₘₐₓ. The third connection segment 5.3, shaped in the form of a circular cylindrical shaft segment 24, serves for stabilizing the plug-type connection and has a diameter d equal to or greater than the width across flats SW.

The width across flats SW of the connection segment 5.1 with a non-circular cross section Q_{5.1} corresponds to the diameter of the inscribed circle of this cross-section, whereas the width across corners E corresponds to the diameter of the circumscribed circle of the respective cross-section.

FIG. 2 shows the body 2 having a second connection zone 6 which corresponds to the first connection zone 3. Adjoining its top end 27, the body 2 comprises the second connection zone 6 equally including three (N=3) connection segments 7.1; 7.2; 7.3, which axially adjoin one another, beginning from the top end 27, and extend over a length L₂. The connection zone 6 is shaped in the form of a shaft segment 11 extending coaxially to the central axis 4. The second connection segment 7.1 having a cross section Q_{7.1} which is in positive engagement with the cross section Q_{5.1} is shaped in the form of a hexagon socket 12 and is equally provided with a width across corners E and a width across flats SW. The second connection segment 7.3 adjoining the second connection segment 7.1 on the side of the top end 27 is shaped in the form of a circular cylindrical bore segment 14, the diameter of which is D14≦E. The second connection segment 7.2 which adjoins the second connection segment 7.1 on the side opposite to the bore segment 14, is equally shaped in the form of a circular cylindrical bore segment 13 with a diameter D13≦SW and dₘᵢₙ≦D13≦dₘₐₓ.

Preferably, the frusto-conical segment 23 representing the first connection segment 5.2 is made from a deformable material, such as, for example, elastomeric or polymeric material such as silicone rubber, polyurethane, etc. so that, in use, as the frusto-conical segment 23 is inserted into the body 2, the frusto-conical segment 23 may deform as it interacts with the circular cylindrical bore segment 13 representing the second connection segment 7.2 so that the overall friction of the wedge-type connection is increased. The remaining connection segments may be made from any bio-compatible material known in the art including, but not limited to, stainless steel, titanium, titanium alloy, etc.

In FIGS. 1 and 2, the first and second connection segments 5.1 and 7.1 form a positive connection relative to rotation about the central axis 4, whereas the axially non-positive connection is accomplished by means of the first connection segment 5.2 shaped in the form of a frusto-conical shaft segment 23 which becomes wedged in the second connection segment 7.2 shaped in the form of a circular cylindrical bore segment 13. The stabilization of the plug-type connection is achieved by the connection, accurately in register with respect to their diameters, of the first connection segment 5.3 shaped in the form of a circular cylindrical shaft segment 24 and the second connection segment 7.3 shaped in the form of a circular cylindrical bore segment 14.

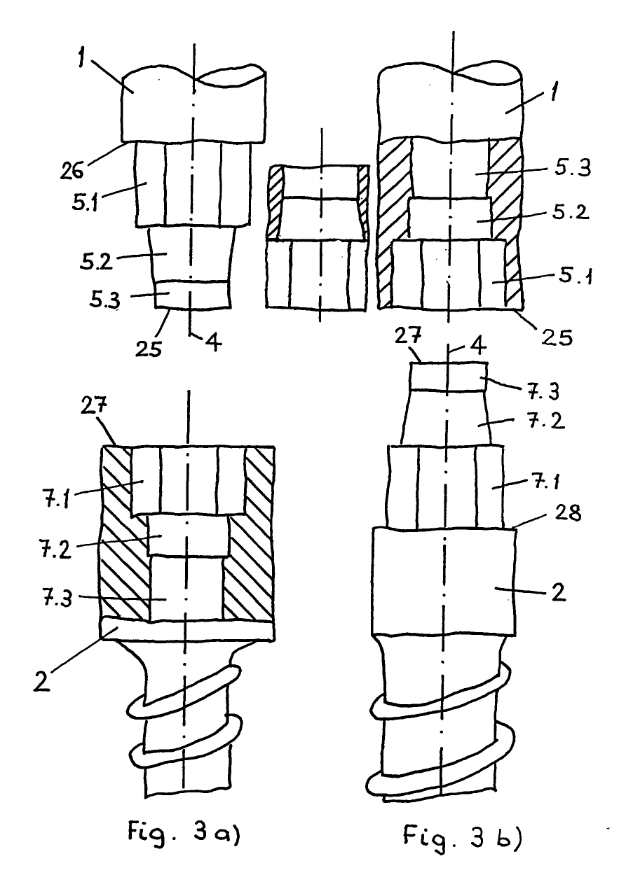

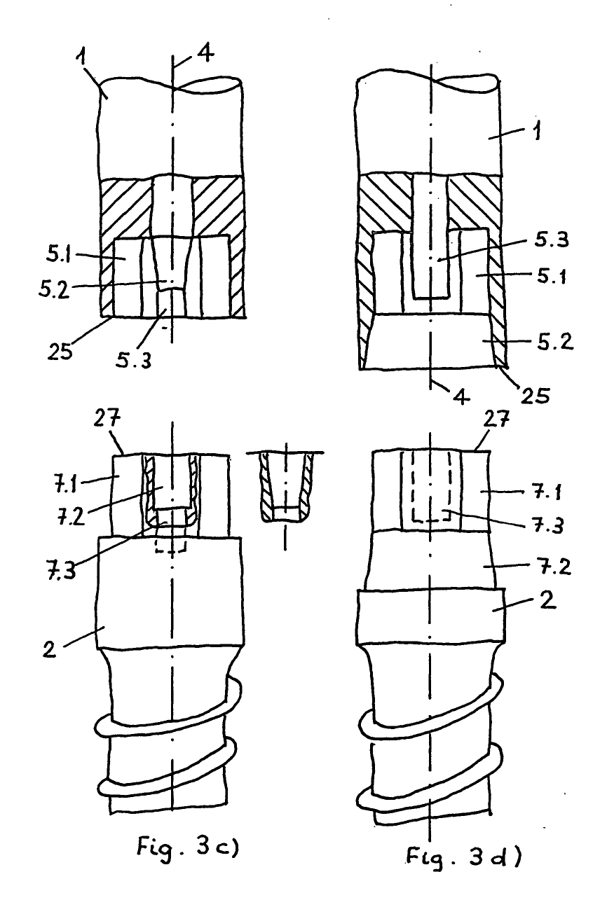

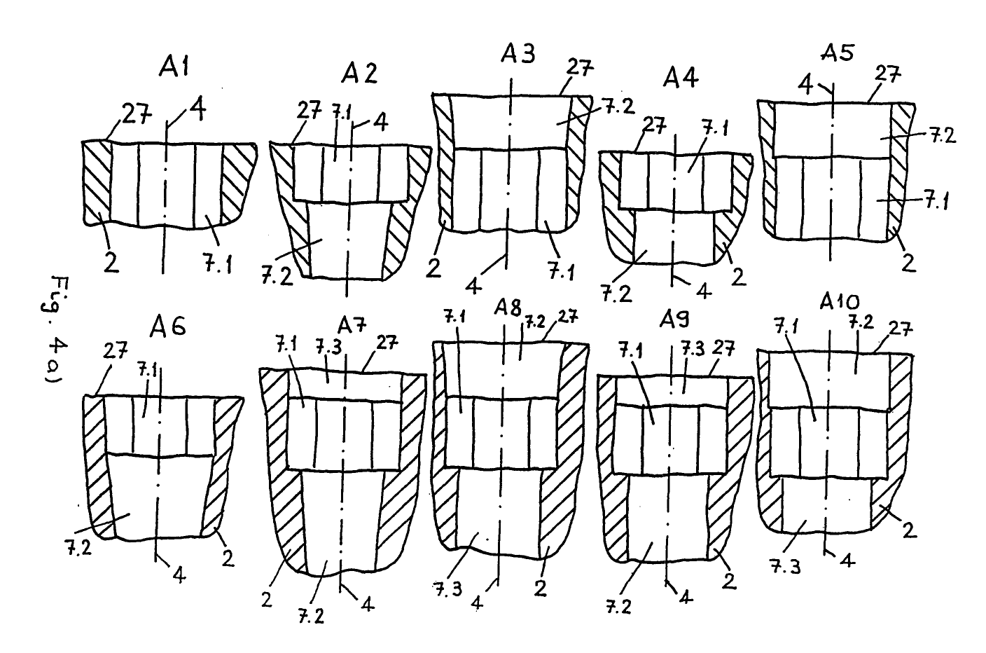

FIGS. 3a) to 3d) show further embodiments of the plug-type connection according to the invention and will be described as follows:

FIG. 3a) The body 1 which, in the present embodiment, represents a screwdriver comprises, in the order mentioned below and beginning at the front end 25, three (M=3) connection segments 5.3; 5.2 and 5.1, extending coaxially to the central axis 4 and axially adjoining one another,
the connection segment 5.1 being shaped in the form of a hexagonal shaft segment with a width across corners E and a width across flats SW;
the connection segment 5.2 being shaped in the form of a frusto-conical shaft segment with dₘₐₓ≦SW; and
the connection segment 5.3 being shaped in the form of a circular cylindrical shaft segment with d≧dₘᵢₙ.

The body 2 which, in the present embodiment, represents a screw comprises, in the order mentioned below and beginning at the top end 27, three (N=3) connection segments 7.1; 7.2 and 7.3, extending coaxially to the central axis 4 and axially adjoining one another,
the connection segment 7.1 being shaped in the form of a hexagonal bore segment with a width across comers E and a width across flats SW;
the connection segment 7.2 being shaped in the form of a circular cylindrical bore segment with d_{7.2}≦SW; and
the connection segment 7.3 being shaped in the form of a circular cylindrical bore segment with d_{7.3}≦d_{7.2}.

FIG. 3b) The body 1 which, in the present embodiment, represents a screwdriver comprises, in the order mentioned below and beginning at the front end 25, three (M=3) connection segments 5.1; 5.2 and 5.3, extending coaxially to the central axis 4 and axially adjoining one another,
the connection segment 5.1 being shaped in the form of a hexagonal bore segment with a width across corners E and a width across flats SW;
the connection segment 5.2 being shaped in the form of a circular cylindrical or frusto-conical bore segment with dₘₐₓ≦SW; and
the connection segment 5.3 being shaped in the form of a circular cylindrical bore segment with d≦dₘᵢₙ.

The body 2 which, in the present embodiment, represents a screw comprises, in the order mentioned below and beginning at the top end 27, three (N=3) connection segments 7.3; 7.2 and 7.1, extending coaxially to the central axis 4 and axially adjoining one another,
the connection segment 7.1 being shaped in the form of a hexagonal shaft segment with a width across corners E and a width across flats SW;
the connection segment 7.2 being shaped in the form of a frusto-conical shaft segment (dₘᵢₙ; dₘₐₓ) with dₘₐₓ≦SW; and
the connection segment 7.3 being shaped in the form of a circular cylindrical shaft segment with d≦dₘᵢₙ.

FIG. 3c) The body 1 which, in the present embodiment, represents a screwdriver, comprises three (M=3) connection segments 5.1; 5.2 and 5.3, extending coaxially to the central axis 4,
the connection segment 5.1 being shaped in the form of a hexagonal bore segment with a width across corners E and a width across flats SW;
the connection segment 5.2 being shaped in the form of a frusto-conical shaft segment, adjoining the connection segment, with dₘₐₓ<SW; and
the connection segment 5.3 being shaped in the form of a circular cylindrical shaft segment, adjoining the front end 25, with d<dₘᵢₙ and the connection segments 5.2 and 5.3 being attached to the body 1 as fixed or axially displaceable shaft segments.

The body 2 which, in the present embodiment, represents a screw, comprises three (N=3) connection segments 7.1; 7.2 and 7.3, extending coaxially to the central axis 4,
the connection segment 7.1 being shaped in the form of a hexagonal shaft segment with a width across corners E and a width across flats SW;
the connection segment 7.2 being shaped in the form of a circular cylindrical bore segment, adjoining the top end 27, with d_{7.2}<SW; and
the connection segment 7.3 being shaped in the form of a circular cylindrical bore segment, adjoining the connection segment 7.2, with d_{7.3}<d_{7.2}.

FIG. 3d) The body 1 which, in the present embodiment, represents a screwdriver, comprises three (M=3) connection segments 5.1; 5.2 and 5.3, extending coaxially to the central axis 4,
the connection segment 5.1 being shaped in the form of a hexagonal bore segment, adjoining the connection segment 5.2, with a width across corners E and a width across flats SW;
the connection segment 5.2 being shaped in the form of a frusto-conical bore segment, adjoining the top end 25, with dₘᵢₙ≧E; and
the connection segment 5.3 being shaped in the form of a circular cylindrical shaft segment with d≦SW and being attached to the body 1 as a fixed or axially displaceable shaft segment.

The body 2 which, in the present embodiment, represents a screw, comprises three (N=3) connection segments 7.1; 7.2 and 7.3, extending coaxially to the central axis 4,
the connection segment 7.1 being shaped in the form of a hexagonal shaft segment, adjoining the top end 27, with a width across corners E and a width across flats SW;
the connection segment 7.2 being shaped in the form of a frusto-conical shaft segment, adjoining the connection segment 7.1, with dₘᵢₙ≧SW; and
the connection segment 7.3 being shaped in the form of a circular cylindrical shaft segment, adjoining the top end 27, with d_{7.3}<SW.

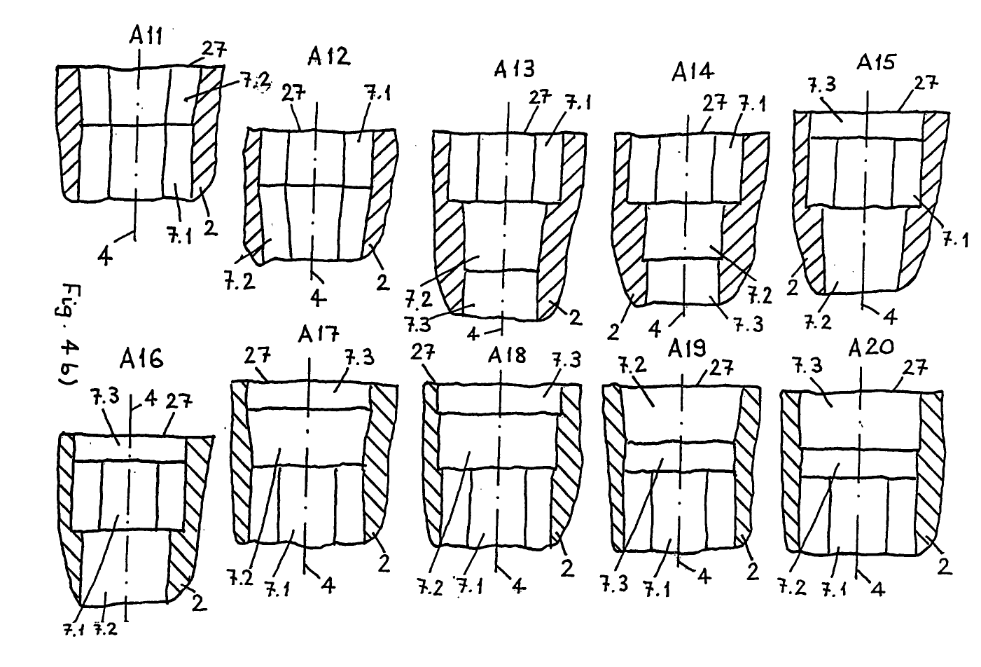

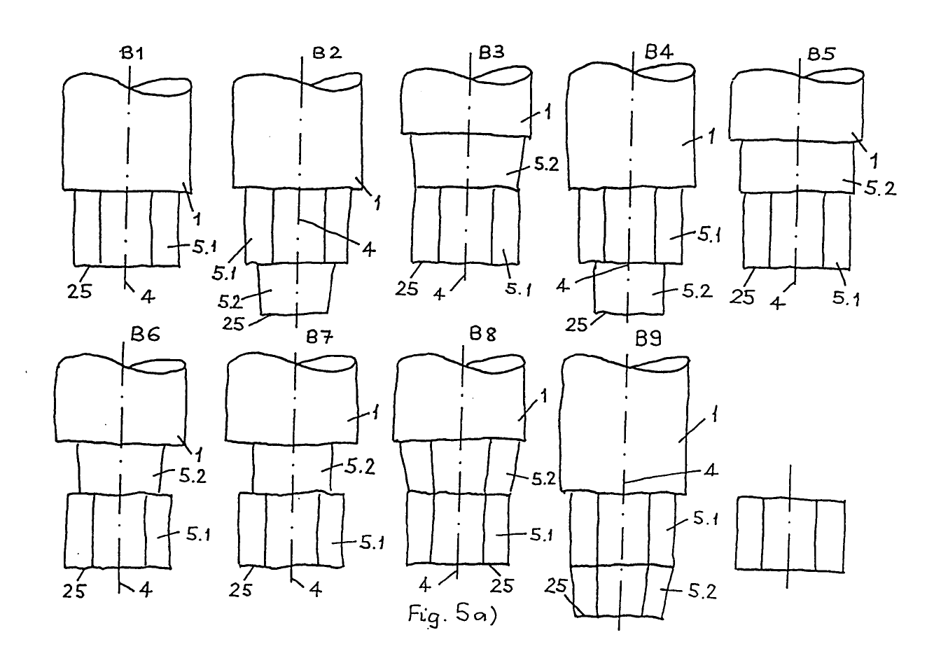

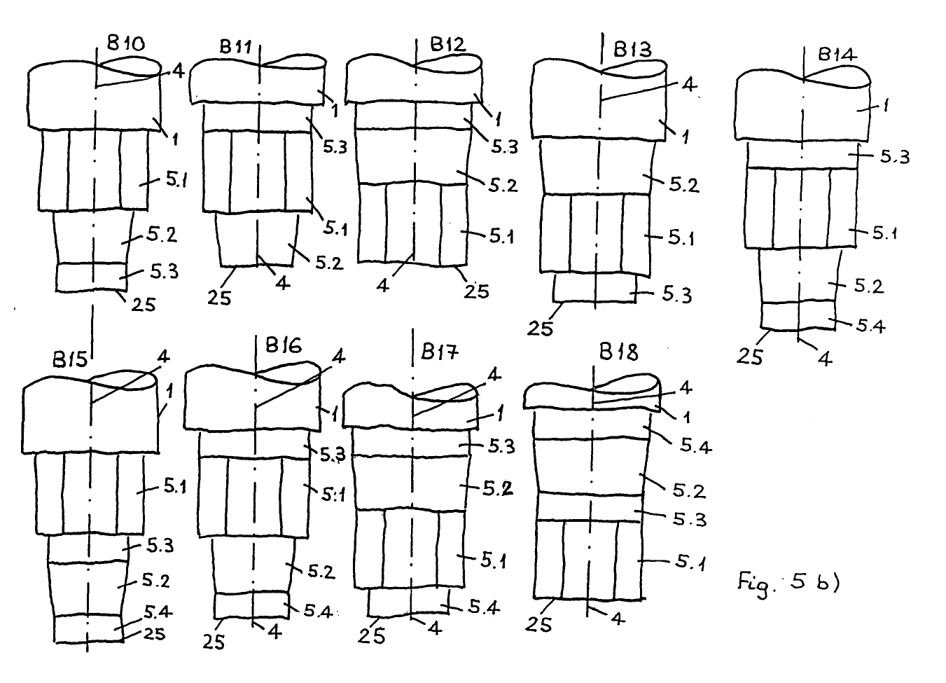

FIGS. 4a), 4b), 5a), and 5b) comprise further embodiments of the plug-type connection according to the invention, realized as bore segments A1 to A20 formed in the body 2 and as shaft segments B1 to B18 formed in the body 1, the respective connection segments being shaped in different forms, as described in the following:

A1: connection segment 7.1 shaped in the form of a hexagon socket;

A2: connection segment 7.1 shaped in the form of a hexagon socket adjoining the top end 27, and connection segment 7.2 shaped in the form of a frusto-conical bore segment, adjoining the connection segment 7.1, with dₘₐₓ≦SW;

A3: connection segment 7.2 shaped in the form of a frusto-conical bore segment, adjoining the top end 27, with dₘᵢₙ≧E, and connection segment 7.1 shaped in the form of a hexagon socket adjoining the connection segment 7.2;

A4: connection segment 7.1 shaped in the form of a hexagon socket adjoining the top end 27, and connection segment 7.2 shaped in the form of a circular cylindrical bore segment, adjoining the connection segment 7.1, with d≦SW;

A5: connection segment 7.2 shaped in the form of a circular cylindrical bore segment, adjoining the top end 27, with d≧E, and connection segment 7.1 shaped in the form of a hexagon socket adjoining the connection segment 7.2;

A6: connection segment 7.1 shaped in the form of a hexagon socket adjoining the top end 27, and connection segment 7.2 shaped in the form of a frusto-conical bore segment, adjoining the connection segment 7.1, with dₘₐₓ≦E;

A7: connection segment 7.3 shaped in the form of a circular cylindrical bore segment, adjoining the top end 27, with a diameter D; connection segment 7.1 shaped in the form of a hexagon socket, adjoining the connection segment 7.3, with E≦D; and connection segment 7.2 shaped in the form of a frusto-conical bore segment, adjoining the connection segment 7.1, with dₘₐₓ≦SW;

A8: connection segment 7.2 shaped in the form of a frusto-conical bore segment, adjoining the top end 27, with dₘᵢₙ≧E; connection segment 7.1 shaped in the form of a hexagon socket, adjoining the connection segment 7.2; and connection segment 7.3 shaped in the form of a circular cylindrical bore segment, adjoining the connection segment 7.1, with d≦SW;

A9: connection segment 7.3 shaped in the form of a circular cylindrical bore segment, adjoining the top end 27, with a diameter D; connection segment 7.1 shaped in the form of a hexagon socket, adjoining the connection segment 7.3, with E≦D; and connection segment 7.2 shaped in the form of a circular cylindrical bore segment, adjoining the connection segment 7.1, with d≦SW;

A10: connection segment 7.3 shaped in the form of a circular cylindrical bore segment, adjoining the top end 27, with a diameter D; connection segment 7.1 shaped in the form of a hexagon socket, adjoining the connection segment 7.3, with E<D; and connection segment 7.2 shaped in the form of a circular cylindrical bore segment, adjoining the connection segment 7.1, with d≧SW;

A11: connection segment 7.2 shaped in the form of a conical hexagon socket adjoining the top end 27, and connection segment 7.1 shaped in the form of a prismatic hexagon socket and adjoined to the connection segment 7.2 by means of an aligning transition;

A12: connection segment 7.1 shaped in the form of a prismatic hexagon socket adjoining the top end 27, and connection segment 7.2 shaped in the form of a conical hexagon socket adjoined to the connection segment 7.1 by means of an aligning transition;

A13: connection segment 7.1 shaped in the form of a hexagon socket adjoining the top end 27; connection segment 7.2 shaped in the form of a frusto-conical bore segment, adjoining the connection segment 7.1, with dₘₐₓ≦SW; and connection segment 7.3 shaped in the form of a circular cylindrical bore segment, adjoining the connection segment 7.2, with d≦dₘₐₓ;

A14: connection segment 7.1 shaped in the form of a hexagon socket adjoining the top end 27; connection segment 7.2 shaped in the form of a circular cylindrical bore segment, adjoining the connection segment 7.1, with D≦SW; and connection segment 7.3 shaped in the form of a circular cylindrical bore segment, adjoining the connection segment 7.2, with d≦D;

A15: identical with A7;

A16: connection segment 7.3 shaped in the form of a circular cylindrical bore segment, adjoining the top end 27, with D≧E; connection segment 7.1 shaped in the form of a hexagon socket, adjoining the connection segment 7.3; and connection segment 7.2 shaped in the form of a circular cylindrical bore segment, adjoining the connection segment 7.1;

A17: connection segment 7.3 shaped in the form of a circular cylindrical bore segment, adjoining the top end 27, with D≧dₘₐₓ; connection segment 7.2 shaped in the form of a frusto-conical bore segment, adjoining the connection segment 7.3; and connection segment 7.1 shaped in the form of a hexagon socket, adjoining the connection segment 7.2, with E≧dₘᵢₙ;

A18: connection segment 7.3 shaped in the form of a circular cylindrical bore segment, adjoining the top end 27, with D>d; connection segment 7.2 shaped in the form of a circular cylindrical bore segment, adjoining the connection segment 7.3, with d<D; and connection segment 7.1 shaped in the form of a hexagon socket, adjoining the connection segment 7.2, with E<d;

A19: connection segment 7.2 shaped in the form of a frusto-conical bore segment, adjoining the top end 27; connection segment 7.3 shaped in the form of a circular cylindrical bore segment, adjoining the connection segment 7.2, with d≦dₘᵢₙ; and connection segment 7.1 shaped in the form of a hexagon socket, adjoining the connection segment 7.3, with E≦d;

A20: connection segment 7.3 shaped in the form of a circular cylindrical bore segment, adjoining the top end 27, with a diameter D; connection segment 7.2 shaped in the form of a circular cylindrical bore segment, adjoining the connection segment 7.3, with d≦D; and connection segment 7.1 shaped in the form of a hexagon socket, adjoining the connection segment 7.3, with E≦d.

In FIG. 5a) the elements B1 to B5 differ from the elements A1 to A5 illustrated in FIG. 4 a) in so far as the respective bore segments are replaced by corresponding shaft segments, the remaining connection segments being shaped in different forms, as described in the following.

B6: connection segment 5.1 shaped in the form of a hexagonal segment adjoining the front end 25, and connection segment 5.2 shaped in the form of a frusto-conical shaft segment, adjoining the connection segment 5.1, with dₘₐₓ>SW;

B7: connection segment 5.1 shaped in the form of a hexagonal segment adjoining the front end 25, and connection segment 5.2 shaped in the form of a circular cylindrical shaft segment, adjoining the connection segment 5. 1, with d≧SW;

B8: differs from the element All only in so far as the bore segments are replaced by corresponding shaft segments;

B9: differs from the element A12 only in so far as the bore segments are replaced by corresponding shaft segments;

B10: connection segment 5.3 shaped in the form of a circular cylindrical shaft segment, adjoining the front end 25, with a diameter d; connection segment 5.2 shaped in the form of a frusto-conical shaft segment, adjoining the connection segment 5.3, with dₘᵢₙ≧d; and connection segment 5.1 shaped in the form of a hexagonal segment, adjoining the connection segment 5.2, with SW≧dₘᵢₙ;

B11: differs from the element A7 only in so far as the bore segments are replaced by corresponding shaft segments;

B12: differs from the element A17 only in so far as the bore segments are replaced by corresponding shaft segments;

B13: differs from the element A8 only in so far as the bore segments are replaced by corresponding shaft segments;

B14: differs from the element B11 only in so far as, adjoining the front end 25, a connection segment 5.4 shaped in the form of a circular cylindrical shaft segment with d≦dₘᵢₙ has been added to the connection segment 5.2;

B15: connection segment 5.4 shaped in the form of a circular cylindrical shaft segment, adjoining the front end 25, with a diameter d; connection segment 5.2 shaped in the form of a frusto-conical shaft segment, adjoining the connection segment 5.4, with dₘᵢₙ≧d; connection segment 5.3 shaped in the form of a circular cylindrical shaft segment, adjoining the connection segment 5.2, with D≧dₘₐₓ; and connection segment 5.1 shaped in the form of a hexagonal segment, adjoining the connection segment 5.3, with SW≧D;

B16: is identical with B14;

B17: differs from the element B12 only in so far as, adjoining the front end 25, a connection segment 5.4 shaped in the form of a circular cylindrical shaft segment with d≦SW has been added to the connection segment 5.1;

B18: differs from the element B12 only in so far as a connection segment 5.3 shaped in the form of a circular cylindrical shaft segment with a diameter D meeting the condition E≦D≦dₘᵢₙ has been inserted between the connection segments 5.1 and 5.2.

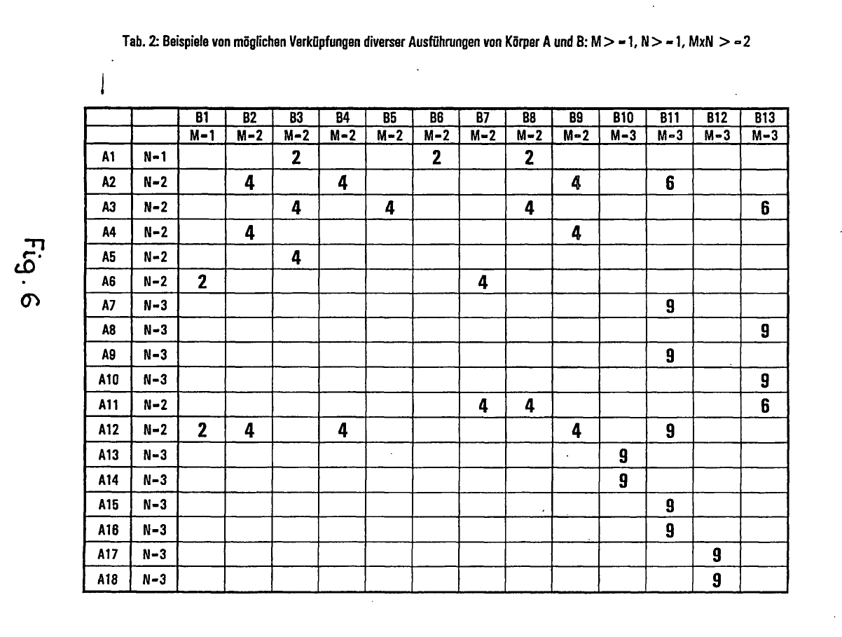

FIG. 6 is a tabular representation of the combinations of the first and second connection segments shaped in the forms of shaft segments B1 to B18 and bore segments A1 to A20, as represented in FIGS. 4a), 4b), 5a), and 5b).

FIGS. 7a) to 7e) show further embodiments of the device according to the invention. These are exemplified by means of a body 2 which is in the form of a screw. A connection segment 7.1 shaped in the form of a hexagonal bore segment adjoins the top end 27 and reaches down to the bottom 29. On the bottom 29, one or two connection segments shaped in the form of shaft segments are arranged which extend towards the top end 27. In FIG. 7a) and 7b), the connection segment 7.2 is shaped in the form of a circular cylindrical shaft segment; in FIG. 7c), the connection segment 7.2 is shaped in the form of a frusto-conical shaft segment; in FIG. 7. d) the connection segment 7.2 is shaped in the form of a frusto-conical shaft segment extending upright from the bottom 29 and adjoined by a connection segment 7.3 shaped in the form of a circular cylindrical shaft segment (d≦dₘᵢₙ); and in FIG. 7 e) the connection segment 7.3 is shaped in the form of a circular cylindrical shaft segment extending upright from the bottom 29 and adjoined by a connection segment 7.2 shaped in the form of a frusto-conical shaft segment (dₘₐₓ≦d).

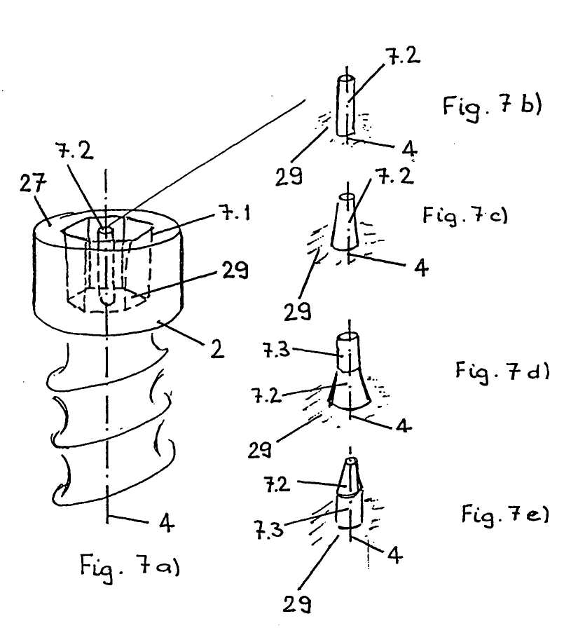

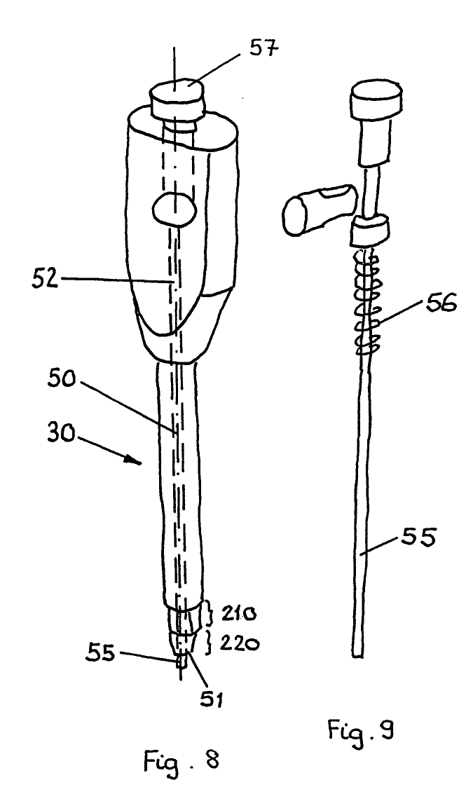

FIGS. 8 and 9 show an embodiment of the device according to the invention in which the body 1 is shaped in the form of a screwdriver 30 which serves for screwing in or screwing out a body 2 (FIG. 2) shaped in the form of a screw. The screwdriver 30 may comprise a longitudinal axis 50, a front end 51 directed towards the body 2 (FIG. 2), and a bore 52 which extends coaxially to the longitudinal axis 50 and is open towards the front end 51. An axially displaceable pin 55 may be positioned within the bore 52 which is pushed within the bore 52 in the direction of the front end 51 by means of a spring 56. The pin 55 may be pushed towards the front end 51 until it abuts against the bottom of the bore segment 11 of the body 2 (FIG. 2). Furthermore, by pushing on its rear end 57, the pin 55 may be displaced at least sufficiently far in the direction of the front end 51 so that the first and second connection segments 5.1 and 7.1, which are in positive engagement with each other and shaped respectively as a hexagon insert bit 210 formed in the screwdriver and as a hexagon socket 12 formed in the screw (FIG. 2), cease to be in mutual engagement and, consequently, also the first connection segment 5.2 (FIG. 1) of the screwdriver 30, shaped in the form of a frusto-conical shaft segment 220, may become completely detached from the bore segment 11 of the body 2 shaped in the form of a screw.

1. A plug-type connection having a central axis for releasably connecting a first body and a second body wherein the first body includes a first connection zone having a longitudinal axis, a length, and a plurality of first connection segments and the second body includes a second connection zone having a longitudinal axis, a length, and a plurality of second connection segments, the connection further comprising:
wherein the plurality of first connection segments are arranged coaxially along the longitudinal axis of the first body, the plurality of first connection segments includes at least one segment with a non-circular cross-section;
wherein the plurality of second connection segments are arranged coaxially along the longitudinal axis of the second body, the plurality of second connection segments include at least one segment with a non-circular cross section sized and configured for rotatively engaging the non-circular cross section of the first connection segment;
wherein the plurality of first connection segments includes at least one segment having an axially tapering cross-section and the plurality of second connection segments includes at least one segment sized and configured to mate with the at least one tapered section, wherein the tapered section of the first connection segment and the mating section of the second connection segment form an axially non-positive engagement for guiding the first body into engagement with the second body and wherein the axially tapering cross-section of the first connection segment is made from a deformable material so that the axially tapering cross section deforms when it mates with the second connection segment.

2. The plug-type connection of claim 1, wherein the segments in the second connection zone axially adjoin one another with respect to the central axis of the second connection zone and differ from one another in shape.

3. The plug-type connection of claim 2, wherein the segments in the first connection zone axially adjoin one another with respect to the central axis of the first connection zone and differ from one another in shape.

4. The plug-type connection of claim 1, wherein the segments in the second connection zone include a shaft segment and a bore segment.

5. The plug-type connection of claim 1, wherein the segments in the first connection zone comprise a shaft segment and a bore segment.

6. The plug-type connection of claim 1, wherein the first connection zone comprises at least two pieces.

7. The plug-type connection of claim 6, wherein at least one segment in the first connection zone is coaxially displaceable relative to the other segments in the first connection zone.

8. The plug-type connection of claim 1, wherein the second connection zone comprises at least two pieces.

9. The plug-type connection of claim 8, wherein at least one segment in the second connection zone is coaxially displaceable relative to the at least one other segment in the second connection zone.

10. The plug-type connection of claim 1, wherein the at least one segment in the first connection zone having the non-circular cross-section and forming the rotationally positive engagement with the at least one segment in the second connection zone, also forms a non-positive engagement with one segment in the second connection zone which is oriented generally parallel to the central axis of the plug-type connection.

11. The plug-type connection of claim 1, wherein at least one segment in the second connection zone forming the rotationally positive engagement with the at least one segment in the first connection zone having the non-circular cross-section, also forms a non-positive engagement with one segment of the first connection zone which is oriented generally parallel to the central axis of the plug-type connection.

12. The plug-type connection of claim 1, wherein the segments forming the positive engagement have cross-sections of complementary geometric shape.

13. The plug-type connection of claim 12, wherein the geometric shape of the complementary cross-sections is selected from the group consisting of polygonal, hexagonal, star-shape, or elliptical.

14. The plug-type connection of claim 1, wherein the number of segments in the first connection zone is greater than the number of segments in the second connection zone.

15. The plug-type connection of claim 14, wherein the number of segments in the first connection zone is greater than the number of segments in the second connection zone by one segment.

16. The plug-type connection of claim 1, wherein the number of segments in the first connection zone and the number of segments in the second connection zone are identical.

17. The plug-type connection of claim 16, wherein the first connection zone and the second connection zone each have three segments.

18. The plug-type connection of claim 17, wherein the segments in the second connection zone comprise a bore segment having a circular cylindrical bore segment with a first diameter, a hexagon socket segment having a first width measured across a pair of opposing flats of the hexagon socket and a second width measured across a pair of adjacent corners of the hexagon socket, and a cylindrical bore segment having a second diameter, the first diameter measuring a distance greater than the second width and the second diameter measuring a distance less than the first width.

19. The plug-type connection of claim 18, wherein the segments in the first connection zone comprise a shaft segment having a circular cylindrical shaft with a first diameter, a hexagonal segment having a first width measured across a pair of adjacent corners of the hexagonal segment and a second width measured across a pair of opposing flats of the hexagonal segment, and the axially tapering shaft segment has an angle between about 2 and 10 degrees, the first diameter in the first connection zone measuring a distance substantially equal to the first diameter of the second connection zone and the tapering shaft segment having a maximum diameter which measures a distance less than the second diameter in the second connection zone.

20. The plug-type connection of claim 17, wherein the segments in the second connection zone comprise a bore segment with a circular cylindrical bore portion having a first diameter, a star-shaped socket segment with six branches having an inside diameter and an outside diameter, and a cylindrical bore segment having a second diameter, the first diameter measuring a distance greater than or equal to the outside diameter of the star shaped socket segment and the second diameter measuring a distance less than or equal to the inner diameter of the star shaped socket segment.

21. The plug-type connection of claim 20, wherein the segments in the first connection zone comprise a shaft segment with a circular cylindrical shaft having a first diameter, a star-shaped segment with six branches having an inside diameter and an outside diameter and a frusto-conical shaft segment having a cone angle of between about 2 and 10 degrees, the first diameter in the first connection zone measuring a distance equal to the first diameter in the second connection zone and the frusto-conical shaft segment having a maximum diameter and a minimum diameter, the maximum diameter measuring a distance greater than the second diameter in the second connection zone and the minimum diameter measuring a distance less than the second diameter in the second connection zone.

22. The plug-type connection of claim 1, wherein at least one of the segments in the second connection zone has a conically tapered form converging in the direction in which the plugging takes place.

23. The plug-type connection of claim 1, wherein at least one of the segments in the first connection zone has a conically tapered form converging in the direction in which the plugging takes place.

24. The plug-type connection of claim 1, wherein at least one of the segments in the second connection zone has a cylindrical form.

25. The plug-type connection of claim 1, wherein at least one of the segments in the first connection zone on the frontal part of the first body has a cylindrical form.

26. The plug-type connection of claim 1, wherein one of the first body and second body is a screw and the other one is a screwdriver.

27. The plug-type connection of claim 26, wherein the screwdriver comprises a longitudinal axis, a front end facing the screw, and a bore which is open towards the front end of the screwdriver and extending coaxially to the longitudinal axis.

28. The plug-type connection of claim 27, wherein an axially displaceable pin is positioned in the bore.

29. The plug-type connection of claim 28, wherein the pin is pushed into the bore by a resilient member.

30. The plug-type connection of claim 29, wherein the pin is capable of being pushed into contact with the screw.

31. The plug type connection of claim 30, wherein pushing the pin into contact with the screw causes the segments in the second connection zone and the segments in the first connection zone which form a positive engagement between the screw and the screwdriver to cease to be in positive engagement.

32. The plug-type connection of claim 31, wherein pushing the pin into contact with the screw causes the screw to disengage from the screwdriver.
